(19) United States
(12) Patent Application Publication
(10) Pub. No.: US 2006/0102175 A1
(43) Pub. Date: May 18, 2006

(54) Inhaler

(76) Inventor: Stephen G. Nelson, San Diego, CA (US)

Correspondence Address: SoCAL IP LAW GROUP LLP, 310 N. WESTLAKE BLVD. STE 120, WESTLAKE VILLAGE, CA, 91362, US

(21) Appl. No.: 11/272,130

(22) Filed: Nov. 9, 2005

Related U.S. Application Data
(60) Provisional application No. 60/629,508, filed on Nov. 18, 2004.

(51) Int. Cl. A62B 7/00; A61M 16/00

(52) U.S. Cl. 128/200.24

(57) ABSTRACT

There is disclosed an apparatus comprising a housing, a phyto material receptacle, a heating element, a heating element switch and a controller. The housing is adapted to be handheld. The phyto material receptacle includes a plurality of cavities. Each cavity is adapted to hold a phyto material unit. The phyto material receptacle is removable from the housing. The heating element is disposed within the housing and proximate one of the plurality of cavities. The heating element switch is adapted to activate the heating element. The controller is adapted to control transfer of energy to the heating element, identify a quantity of unused phyto material units held by the phyto material receptacle, and control the heating element switch.

### RELATED APPLICATION INFORMATION

This application claims priority from Provisional Application No. 60/629,508, filed Nov. 18, 2004, which is incorporated herein by reference.

### NOTICE OF COPYRIGHTS AND TRADE DRESS

A portion of the disclosure of this patent document contains material which is subject to copyright protection. This patent document may show and/or describe matter which is or may become trade dress of the owner. The copyright and trade dress owner has no objection to the facsimile reproduction by any one of the patent disclosure as it appears in the Patent and Trademark Office patent files or records, but otherwise reserves all copyright and trade dress rights whatsoever.

### BACKGROUND OF THE INVENTION

1. Field of the Invention

The present invention relates to inhaling devices.

2. Description of the Related Art

Herbs and botanicals have been used for thousands of years by both ancient and modern civilizations. Aromatherapy, herbology, and phyto-inhalation products are popular among many people.

At least a billion people worldwide smoke tobacco and other phyto materials. However, because of health considerations or various laws and regulations, smoking is now prohibited in many airplanes, restaurants and other public places.

Products are now being introduced to deliver the pleasures of smoking without polluting the environment. Examples of such products include nicotine chewing gum and patches.

### DESCRIPTION OF THE DRAWINGS

FIG. 1A is a perspective view of the first embodiment of the volatizing or vaporizing unit.

FIG. 1B is a perspective view of the first embodiment of the volatizing or vaporizing unit.

FIG. 2A is a perspective view of the sliding mouthpiece.

FIG. 2B is a perspective view of the rotating mouthpiece.

FIG. 3A is a perspective view of four AA batteries.

FIG. 3B is a perspective view of a four AA battery pack.

FIG. 3C is a perspective view of a 9-Volt battery.

FIG. 3D is a perspective view of a proprietary battery pack.

FIG. 4A is a perspective view of a constituent disk.

FIG. 4B is a perspective view of a constituent disk with pattern identification.

FIG. 4C is a perspective view of a constituent disk with electrical identification.

FIG. 4D is a perspective view of a constituent disk with integrated heating elements.

FIG. 5 is a perspective view of the heating chamber and exhaust chamber.

FIG. 6 is a perspective view of the second embodiment of the volatizing or vaporizing unit.

FIG. 7 is a perspective view of the third embodiment of the volatizing or vaporizing unit.

FIG. 8A is a perspective view of the fourth embodiment of the volatizing or vaporizing unit.

FIG. 8B is a perspective view of the fourth embodiment of the volatizing or vaporizing unit.

FIG. 8C is a section view of the fourth embodiment of the volatizing or vaporizing unit.

### DETAILED DESCRIPTION OF THE INVENTION

Throughout this description, the embodiments and examples shown should be considered as exemplars, rather than limitations on the apparatus and methods of the present invention.

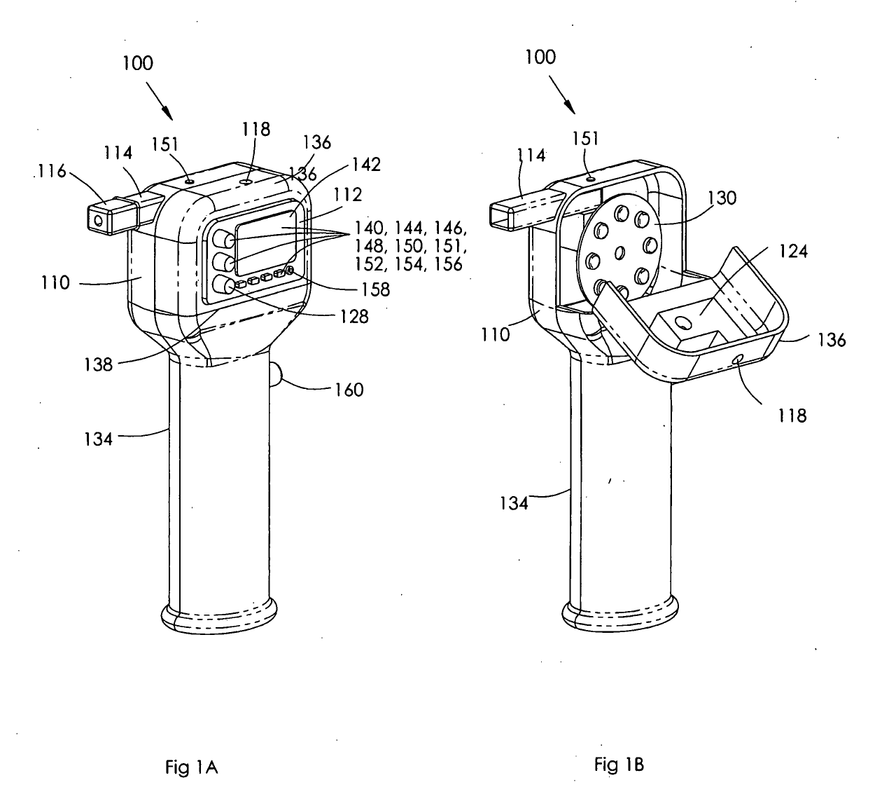

Referring to FIGS. 1A & 1B, the first embodiment of a volatizing or vaporizing unit 100 comprises a frame 110, a control panel 112, a mouthpiece 114, a sanitary disposable sleeve 116, an air inlet 118, a heating chamber 120, a heating element 122, an exhaust chamber 124, a battery pack 126, a power switch 128, and a consumable disk 130 further comprising a constituent 132 to be volatized or vaporized for inhalation.

The frame 110 further comprises a handle 134 and a lid 136. The frame 110 may be made from injection molded plastic, formed sheet metal, carbon fiber composites, or any similar material and processes known to one skilled in the art of making hand held devices. The handle 134 is shaped to provide a comfortable and ergonomic grip. The handle 134 may have finger grips or may be smooth. The shape may be cylindrical, oval, or custom contoured to fit many different sized hands as well as left and right hands. The lid 136 opens to provide access to the heating chamber 120 and heating element 122. The lid 136 may open in a rotational manner via a hinge 138 or in a sliding motion (not shown).

The control panel 112 comprises a temperature setting 140 to control the temperature of the heating element 122. The temperature setting 140 could be in the form of a variety of options. It could be a constant setting or it could have the ability to vary. The temperature setting 140 could be a dial, which is manually turned to increase or decrease the temperature of the heating element 122, it could be a digital gauge with buttons to increase or decrease the temperature, or it could be automatically controlled via internal circuitry embedded in the frame 110 which reads a constituent identification 176 located on the, disk 130. The control panel 112 may further comprise a digital readout 142 for communicating information to the individuals or patients. The digital readout 142 may comprise indicators including, but not limited to, a battery life indicator 144, a number of uses remaining on the disk 146, a date and time indicator 148, and a constituent type indicator 150, which may be manually set or may automatically update by reading the constituent identification 176 located on the disk. The digital readout 142 may further comprise a power on indicator 151 in the form of a small light or LED. The power on indicator may also be located in the frame 110 or the lid 136. The digital readout 142 may further comprise a date and time last used indicator 152 and an alarm for next use indicator 154 for the purpose of dispensing time sensitive medicines to the individuals or patients. The control panel 112 may further comprise a heating timer 156 to control amount of time the heating element 122 is energized. The heating timer 156 could be in the form of a variety of options. It could be a constant setting or it could have the ability to vary. The heating timer 156 could be a dial, which is manually turned to increase or decrease the duration of time the heating element 122 is energized, it could be a digital gauge with buttons to increase or decrease the time, or it could be automatically controlled via internal circuitry embedded in the frame 110 which reads a constituent identification 176 located on the disk 130. The control panel 112 further comprises an electrical power port 158 for recharging the battery pack 126 or for operating without battery power. The electrical power port may also be located on the handle 134 or the frame 110.

The power switch 128 may be a rocker switch, a momentary rocker switch, a push button switch, a momentary push button switch. The power switch may be located on the control panel 112, the frame 110 (not shown), or the handle 134 (not shown). Alternatively, the volatizing or vaporizing unit 100 may be powered on by sliding or opening the mouthpiece 114. When the power switch 128 is activated, the volatizing or vaporizing unit 100 is put into a “powered on” state, whereby it is ready to be function as described. When the power switch 128 is deactivated, the volatizing or vaporizing unit 100 is put into a “powered off’ state. With any of the aforementioned on-off switch 158 configurations, the volatizing or vaporizing unit 100 may have a power save mode whereby the volatizing or vaporizing unit 100 may power down after a predetermined time of inactivity.

The frame 110 further comprises heater switch 160. The heater switch 160 may be a rocker switch, a momentary rocker switch, a push button switch, a momentary push button switch, or a momentary trigger switch similar to gun. Alternatively, the heater switch 160 may be internal to the volatizing or vaporizing unit 100, which is activated when the individual or patient applies suction to the mouthpiece 114 by inhaling on the mouthpiece 114. With any of the aforementioned momentary heater switch 160 configurations, the volatizing or vaporizing unit 100 may have a power save mode whereby the volatizing or vaporizing unit 100 may sustain energy to the heating element 122 for a predetermined time or automatically sustain energy to the heating element 122 for a time related to the constituent identification 176.

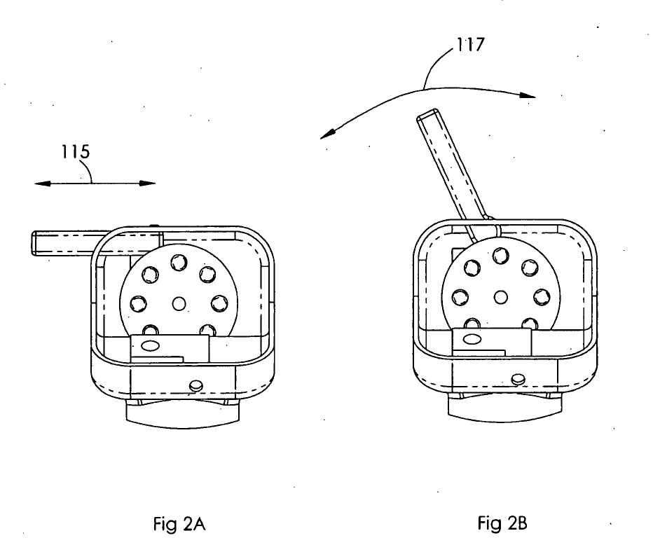

Referring to FIGS. 2A & 2B, the mouthpiece 114 may be made from injection molded plastic, formed sheet metal, carbon fiber composites, or any similar material and processes known to one skilled in the art of making mouthpieces. The mouthpiece 114 comprises a mouthpiece inlet 162, an internal passageway 164, and a mouthpiece exit 166. An optional sanitary disposable sleeve 116 may be placed on the exit of the mouthpiece 114 to allow multiple individuals or patients to use the device without communicating germs or saliva. The mouthpiece 114 may slide open and closed along sliding path 115 or may rotate open or closed along rotating path 117.

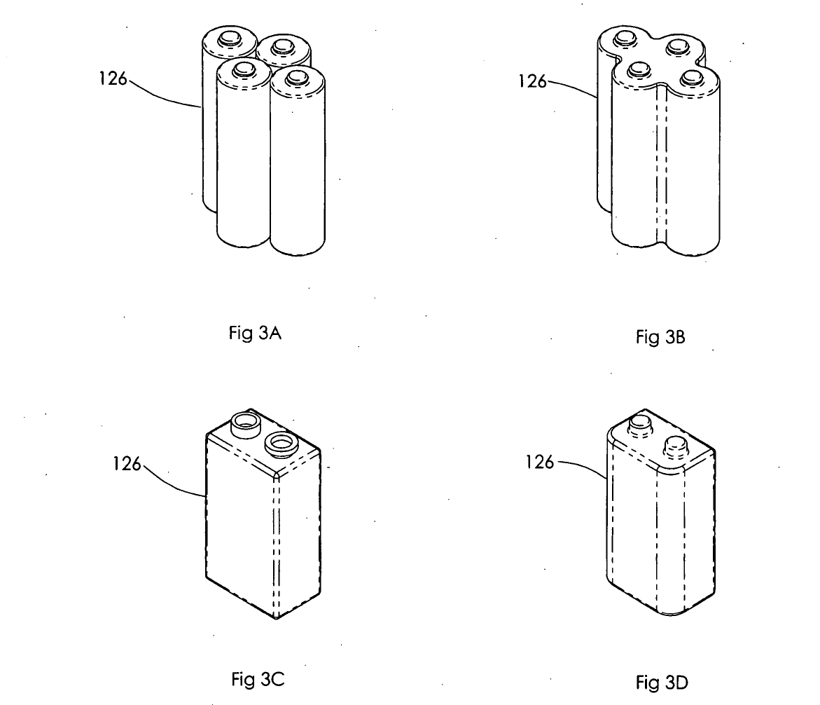

Referring to FIGS. 3A-D, the battery pack 126 may be a proprietary shape to ensure proper installation, it may also be rechargeable type of nickel metal hydride, known as Ni-mh, or lithium ion, know as Li-ion, or other types known to one of ordinary skill in the art of rechargeable batteries. The battery pack 126 may be interchangeable with one or more disposable alkaline batteries including, but not limited to, AA, 9-volt, C-Cell, or rechargeable batteries with the aforementioned form factor.

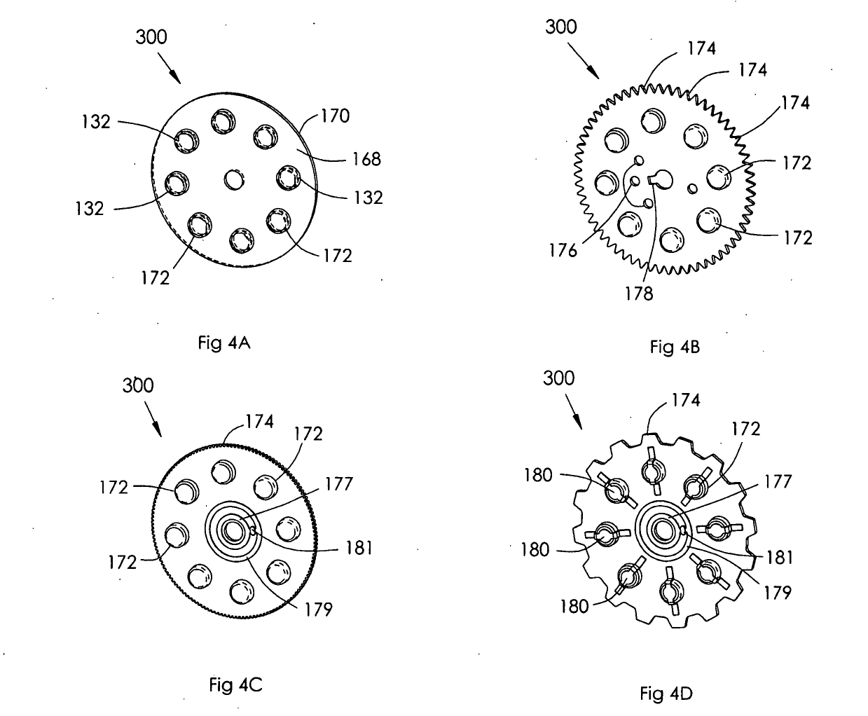

Referring to FIGS. 4A-D, the disk 130 may be formed of a metal mesh, porous, or air permeable, material such as ceramic or sintered metal, or any other material which is heat resistant and portions of which will allow air, gas, or liquid fluids to pass through. The disk 130 may comprise a top surface or layer 168, a bottom surface or layer 170. The bottom layer may have dimples or cups 172 formed to hold a constituent 132 to be exposed to heat. The dimples or cups 172 may further comprise small holes or openings (not shown), which will allow air, gas or liquid fluids to pass through. The disk 130 may further comprise ratchet teeth 174 on the exterior of the disk or the interior (not shown) for the purpose of rotating and locating the dimples or cups 172 with respect to the heating chamber 120. Referring again to FIGS. 4A-D, the ratchet teeth 174 are shown with varying shapes to illustrate several options for rotating and locating the dimples or cups 172 with respect to the heating chamber 120. The ratchet teeth 174 shown are no way intended to limit the shape to the options illustrated, but include all equivalent shapes, which may be used for rotating the disk 130. The disk may comprise a constituent identification 176 in the form of holes in a pattern or via resistance shown with an inner trace 177 and an outer trace 179 with an identification resistor 181 connecting the traces. The constituent identification 176 may be read by the volatization or vaporization unit 100 for the purposes of controlling the temperature and time heat is applied to the constituent 132. The disk may further comprise a key feature 178 in the center for locating and rotating the disk. The dimples or cups 172 may further comprise air permeable integrated heating elements 122 used for applying heat to the constituent 132. The integrated heating elements 122 may comprise of an electrical resistive material including, but not limited to an aluminum alloy, a ferrous alloy, carbon filament yarns, and equivalents thereof. The integrated heating elements 122 may be designed to provide specific temperatures to the constituent 132 depending on the type of constituent 132 in the disk. When the heater switch 160 is activated, electrical energy is applied to the outer trace and inner trace of the integrated heating element 122 causing the integrated heating element 122 to reach a temperature sufficient to volatize or vaporize the constituent 132 within the dimple or cup of the disk.

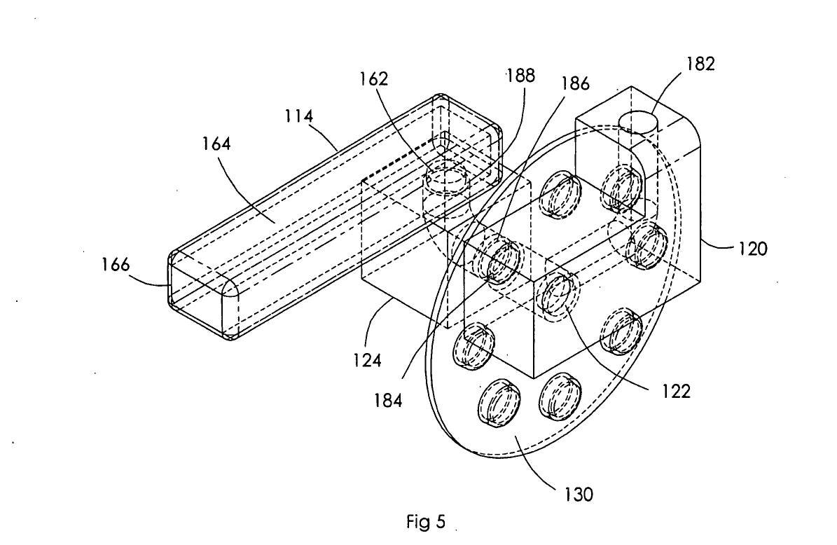

Referring to FIG. 5, the heating chamber 120 and exhaust chamber 124 may be made from a heat resistant material with insulating properties. The material may be ceramic, sintered metal, or a composite of heat resistant material such as metal coated with an insulating material or fabric. The heating chamber 120 comprises a heating inlet 182 and a heating exit 184. The exhaust chamber 124 comprises an exhaust inlet 186 and an exhaust exit 188. The heating inlet 182 of the heating chamber 120 allows ambient air to be drawn into the heating chamber 120 to be heated. The dimple or cup 172 of the disk 130 is located in sufficient proximity to the heating element 122 in order to be heated to the point of volatization or vaporization of the constituent 132 within the dimple or cup. The heating exit 184 of the heating chamber 120 is in close proximity or in direct contact the dimple or cup 172 on the bottom surface or layer 170 side of the disk 130 to focus the heat from the heating element 122 to the constituent 132 within the dimple or cup. The exhaust inlet 186 of the exhaust chamber 124 is in close proximity or in direct contact of the top surface or layer 169 of the disk 130 opposite from the dimple or cup. The exhaust chamber 124 exhaust exit 188 is positioned to create a seal with the mouthpiece inlet 162. During operation the individual or patient initiates the heating of the heating element 122 by pressing or activating the heater switch 160 while applying suction at the mouthpiece exit 166 of the mouthpiece 114 by inhaling slightly. The suction from inhaling draws ambient air into the heating inlet 182 into the heating chamber 120, through the dimple or cup 172 in the disk 130, whereby the air mixes with the volatized or vaporized constituent 132 and is drawn through the exhaust inlet 186, out the exhaust exit 188 of the exhaust chamber 124, into the mouthpiece inlet 162 of the mouthpiece 114, through the mouthpiece exit 166 and into the individual's or patient's lungs. Alternatively, the heater switch may be activated by an on demand vacuum or suction operated switching mechanism (not shown), whereby when the individual or patient applies suction at the mouthpiece exit 166, the heater switch 160 is activated initiating the delivery of the volatized or vaporized constituent 132 into the lungs. The mouthpiece 114 may also be an integrated mouthpiece 114 and exhaust chamber 124 (not shown). The heating element 122 may be made of a resistive electrical heating element 122 similar to an electrocautery surgical tool or an electric stove heating element 122.

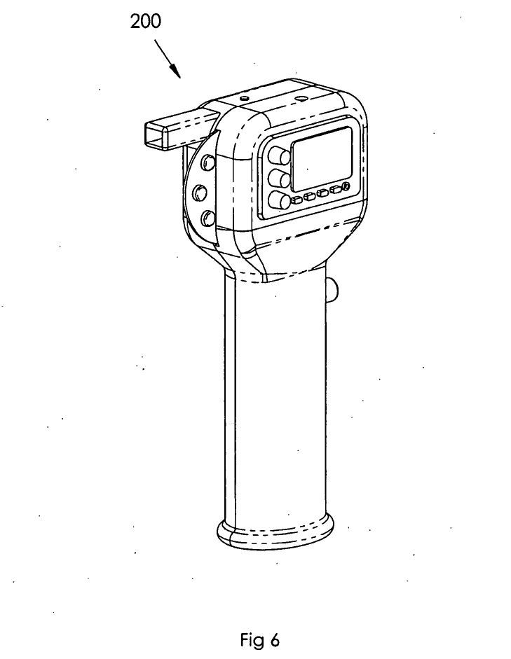

Referring to FIG. 6, a second embodiment of the volatizing or vaporizing unit 200 comprises a frame 110, a control panel 112, a mouthpiece 114, a sanitary disposable sleeve 116, an air inlet 118, a heating chamber 120, a heating element 122, an exhaust chamber 124, a battery pack 126, a power switch 128, a heater switch 160, a consumable disk 130 containing a constituent 132 to be volatized or vaporized for inhalation, and a side opening. The disk 130 is inserted and removed via a side opening. The disk 130 rotates as described hereinabove.

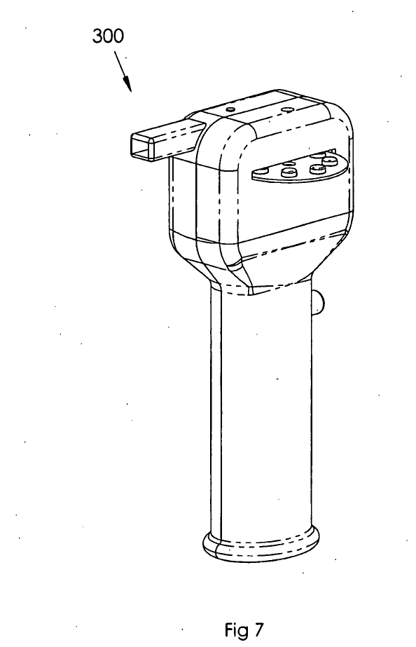

Referring to FIG. 7, a third embodiment of the volatizing or vaporizing unit 300 comprises a frame 110, a control panel 112, a mouthpiece 114, a sanitary disposable sleeve 116, an air inlet 118, a heating chamber 120, a heating element 122, an exhaust chamber 124, a battery pack 126, a heater switch 160, a consumable disk 130 containing a constituent 132 to be volatized or vaporized for inhalation, and a side opening. The disk 130 is inserted and removed via a side opening. The disk 130 is inserted and removed via a side opening, whereby the disk 130 is positioned horizontal or perpendicular to the position of the first and second embodiments. The disk 130 rotates as described hereinabove.

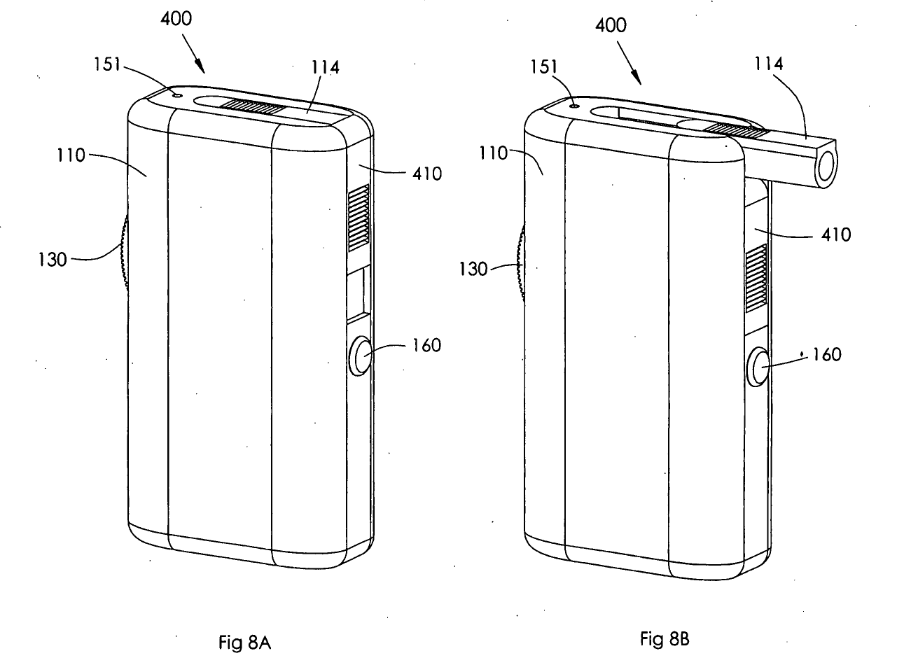

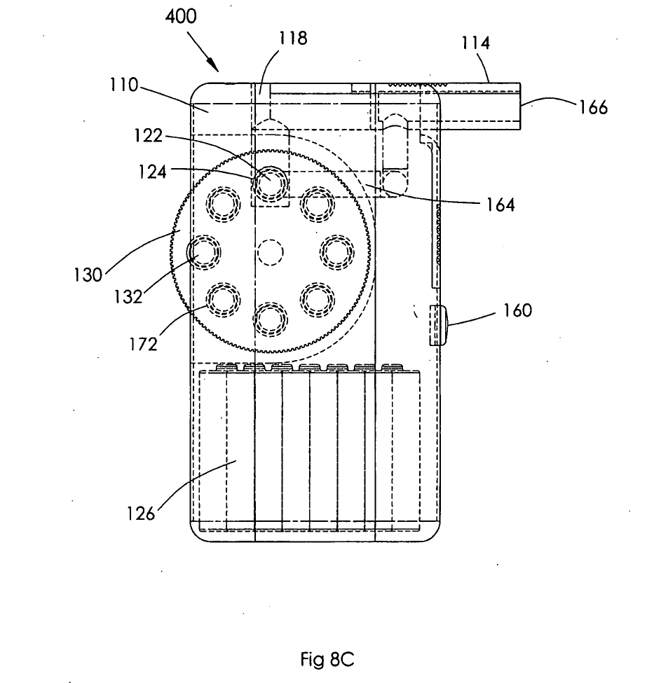

Referring to FIGS. 8A & 8B, a fourth embodiment of the volatizing or vaporizing unit 400 comprises a frame 110, a mouthpiece 114, a sliding door 410, a consumable disk 130 containing a constituent 132 to be volatized or vaporized for inhalation, a heater switch 160, and several features not shown including an air inlet 118, a heating chamber 120, a heating element 122, an exhaust chamber 124, a battery pack 126, a power switch 128, and a side opening. The disk 130 is inserted and removed via a side opening. The disk 130 is inserted and removed via a side opening, whereby the disk 130 is positioned horizontal or perpendicular to the position of the first and second embodiments. The disk 130 rotates as described hereinabove.

Referring to FIG. 8A, the fourth embodiment the volatizing or vaporizing unit 400 is shown in the closed position whereby the sliding door 410 covers the end of the mouthpiece 114. Referring to FIG. 8B, the fourth embodiment 400 is shown in the open position whereby the sliding door 410 is moved toward the heater switch 160 allowing the mouthpiece 114 to slide into the open position as shown. When the sliding door 410 is in the open position it may activate the power to the volatizating or vaporizing unit 400. When the individual or patient pressed the heater switch 160, the heating element 122 volatizes or vaporizes the constituent 132 allowing the individual or patient to inhale the constituent 132.

Referring to FIG. 8C, the fourth embodiment the volatizing or vaporizing unit 400 is shown with the air inlet 118, the heating chamber 120, the heating element 122, the exhaust chamber 124, the battery pack 126, the disk 130, the constituent 132 located in the dimples or cups 172, and the internal passageway 164 in dashed lines. As described hereinabove, during operation the individual or patient initiates the heating of the heating element 122 by pressing or activating the heater switch 160 while applying suction at the mouthpiece exit 166 of the mouthpiece 114 by inhaling slightly. The suction from inhaling draws ambient air into the heating inlet 182 into the heating chamber 120, through the dimple or cup 172 in the disk 130, whereby the air mixes with the volatized or vaporized constituent 132 and is drawn through the exhaust inlet 186, out the exhaust exit 188 of the exhaust chamber 124, into the mouthpiece inlet 162 of the mouthpiece 114, through the mouthpiece exit 166 and into the individual's or patient's lungs. Alternatively, the heater switch may be activated by an on demand vacuum or suction operated switching mechanism (not shown), whereby when the individual or patient applies suction at the mouthpiece exit 166, the heater switch 160 is activated initiating the delivery of the volatized or vaporized constituent 132 into the lungs.

For any of the embodiments of the volatizing or vaporizing unit 100, 200, 300 or 400 described hereinabove, the disk 130 rotates or ratchets a predetermined number of degrees in order to index the dimples or cups 172 to align with the heating chamber 120. The disk 130 may be automatically rotated via the control panel 112 electronics or manually by the individuals or patients.

Closing Comments

The foregoing is merely illustrative and not limiting, having been presented by way of example only. Although exemplary embodiments of the invention have been shown and described, it will be apparent to those having ordinary skill in the art that changes, modifications, and/or alterations may be made, none of which depart from the spirit of the present invention. All such changes, modifications and alterations should therefore be seen as within the scope of the present invention.

Although many of the examples presented herein involve specific combinations of method acts or system elements, it should be understood that those acts and those elements may be combined in other ways to accomplish the same objectives. With regard to flowcharts, additional and fewer steps may be taken, and the steps as shown may be combined or further refined to achieve the methods described herein. Acts, elements and features discussed only in connection with one embodiment are not intended to be excluded from a similar role in other embodiments.

For any means-plus-function limitations recited in the claims, the means are not intended to be limited to the means disclosed herein for performing the recited function, but are intended to cover in scope any means, known now or later developed, for performing the recited function.

As used herein, “plurality” means two or more.

As used herein, a “set” of items may include one or more of such items.

As used herein, whether in the written description or the claims, the terms “comprising”, “including”, “carrying”, “having”, “containing”, “involving”, and the like are to be understood to be open-ended, i.e., to mean including but not limited to. Only the transitional phrases “consisting of” and “consisting essentially of”, respectively, are closed or semi-closed transitional phrases with respect to claims.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

As used herein, “and/or” means that the listed items are alternatives, but the alternatives also include any combination of the listed items.

### FIGURE LIST

First embodiment of a volatizing volatizing or vaporizing unit 100
Frame 110
Control panel 112
Mouthpiece 114
Sliding path 115
Sanitary disposable sleeve 116
Rotating path 117
Air inlet 118
Heating chamber 120
Heating element 122
Exhaust chamber 124
Battery pack 126
Power switch 128
Disk 130
Constituent 132
Handle 134
Lid 136
Hinge 138
Temperature setting 140
Digital readout 142
Battery life indicator 144
Number of uses remaining on the disk 146
Date and time indicator 148
Constituent type indicator 150
Power on indicator 151
Date and time last used indicator 152
Alarm for next use indicator 154
Heating timer 156
Electrical power port 158
Heater switch 160
Mouthpiece inlet 162
Internal passageway 164
Mouthpiece exit 166
Top surface or layer 168
Bottom surface or layer 170
Dimples or cups 172
Ratchet teeth 174
Constituent identification 176
Inner trace 177
Key feature 178
Outer trace 179
Integrated heating elements 180
Identification resistor 181
Heating inlet 182
Heating exit 184
Exhaust inlet 186
Exhaust exit 188
Second embodiment of a volatizing volatizing or vaporizing unit 200
Third embodiment of a volatizing volatizing or vaporizing unit 300
Fourth embodiment of a volatizing volatizing or vaporizing unit 400
Sliding door 410

It is claimed:

1. An apparatus comprising:
a housing adapted to be handheld
a phyto material receptacle including a plurality of cavities, each cavity adapted to hold a phyto material unit, the phyto material receptacle removable from the housing
a heating element disposed within the housing and proximate one of the plurality of cavities
a heating element switch adapted to activate the heating element
a controller adapted to
control transfer of energy to the heating element
identify a quantity of unused phyto material units held by the phyto material receptacle
control the heating element switch.

2. The apparatus of claim 1 wherein the phyto material receptacle includes a phyto type identifier.

3. The apparatus of claim 1 wherein the heating element is integral with the cavity.

4. The apparatus of claim 1 further comprising an indicator adapted to identify a time the heating element was activated.

5. The apparatus of claim 1 wherein the controller is further adapted to control transfer of energy to the heating element based on the phtyo type identifier and control the heating element switch based on the phyto type identifier.

6. The apparatus of claim 1 further comprising an alarm adapted to provide a next use reminder.

7. The apparatus of claim 6 wherein the controller is further adapted to control the alarm based on the time the heating element was activated.

8. The apparatus of claim 1 further comprising a power switch and a mouthpiece, wherein the mouthpiece is adapted to slide relative to the housing and activate the power switch when slid relative to the housing, wherein the power switch is electrically coupled with the heating element.

9. The apparatus of claim 2 wherein the phyto type identifier has an inner trace, an outer trace and a resistor.

10. The apparatus of claim 1 wherein the heating element switch is adapted to activate the heating element when suction is applied to the mouthpiece.

11. The apparatus of claim 1
wherein the phtyo material receptacle includes a phyto type identifier
wherein the heating element is integral with the cavity
wherein the controller is further adapted to control transfer of energy to the heating element based on the phyto type identifier and control the heating element switch based on the phyto type identifier.

12. The apparatus of claim 11 further comprising an indicator adapted to identify the time the heating element was activated.

13. An inhaler device comprising:
a phyto material receptacle including a plurality of cavities
a heating element disposed proximate one of the plurality of cavities
a heating element switch adapted to activate the heating element
a processor
a memory coupled with the processor
a storage medium having instructions stored thereon which when executed cause the inhaler device to perform actions comprising
controlling transfer of energy to the heating element
identifying a quantity of unused phyto material units held by the phyto material receptacle
controlling the heating element switch.

14. The inhaler device of claim 13 wherein the phyto material holder includes a phyto type identifier and the storage medium has further instructions stored thereon which when executed cause the inhaler device to perform actions comprising controlling transfer of energy to the heating element based on the phyto type identifier and controlling the heating element switch based on the phyto type identifier.

15. The inhaler device of claim 14 further comprising an alarm adapted to provide a next use reminder, wherein the storage medium has further instructions stored thereon which when executed cause the inhaler device to perform actions comprising controlling the alarm based on a time the heating element was activated.

16. A storage medium having instructions stored thereon which when executed by a processor will cause the processor to perform actions comprising:
controlling transfer of energy to a heating element of an inhaler device
identifying a quantity of unused phyto material units held by a phyto material receptacle of the inhaler device
activating a heating element switch of the inhaler device, the heating element switch electrically coupled with the heating element.

17. The storage medium of claim 16 having further instructions stored thereon which when executed by a processor will cause the processor to perform actions comprising controlling transfer of energy to the heating element based on a phyto type identifier of the phyto material receptacle.

18. The storage medium of claim 16 having further instructions stored thereon which when executed by a processor will cause the processor to perform actions comprising controlling the heating element switch based on the phyto type identifier.

19. The storage medium of claim 16 having further instructions stored thereon which when executed by a processor will cause the processor to perform actions comprising recording a time the heating element switch was activated.

20. The storage medium of claim 19 having further instructions stored thereon which when executed by a processor will cause the processor to perform actions comprising controlling an alarm of the inhaler based on the time the heating element switch was activated.
